(19) United States
(12) Patent Application Publication
(10) Pub. No.: US 2010/0191224 A1
(43) Pub. Date: Jul. 29, 2010

(54) MAGNETIC DETENT MECHANISM FOR MEDICAL INSTRUMENTS

(75) Inventor: Ken Butcher, Conyers, GA (US)

Correspondence Address: KNOBBE MARTENS OLSON & BEAR LLP, 2040 MAIN STREET, FOURTEENTH FLOOR, IRVINE, CA, 92614, US

(73) Assignee: C.R. BARD, INC., Covington, GA (US)

(21) Appl. No.: 12/637,564

(22) Filed: Dec. 14, 2009

Related U.S. Application Data
(60) Provisional application No. 61/122,686, filed on Dec. 15, 2008.

(51) Int. Cl. A61B 17/00

(52) U.S. Cl. 606/1

(57) ABSTRACT

A medical instrument includes a channel, a first interacting structure, a slider, and a control member. The first interacting structure can extend along at least a portion of the channel. At least a portion of the slider can also be disposed in the channel, so as to move in the channel. The slider can additionally have a first attraction portion. The control member can be rotatably coupled to the slider, and have a second interacting structure and a second attraction portion. The second interacting structure can engage the first interacting structure so as to move the slider in the channel when the control member is rotated. The second attraction portion can be disposed such that a distance between the second attraction portion and the first attraction portion changes as the control member is rotated relative to the slider.

### PRIORITY INFORMATION

This application claims the priority benefit under 35 U.S.C. §119(e) to U.S. Provisional Patent Application Ser. No. 61/122,686 (filed Dec. 15, 2008), the entirety of which is hereby expressly incorporated by reference herein.

### BACKGROUND OF THE INVENTION

1. Field of the Invention

The invention relates to medical instruments that provide tactile feedback to an operator. More particularly, magnets are used to provide the tactile feedback.

2. Description of the Related Art

Preferably, human-operated medical instruments provide some form of visible, audible, and or tactile feedback during use. This feedback can indicate a state of the instrument and/or a state of what the instrument is operating upon such as the patient. Prior art instruments typically use mechanisms such as springs, mechanical stops, or ratchets to provide feedback.

### SUMMARY OF THE INVENTION

The prior art instruments are unsatisfactory in a number of ways. For example, the mechanical feedback devices such as springs, mechanical stops, or ratchets can wear out over time, and thus are not always reliable. As another example, these mechanisms may be difficult to modify to provide a specific desired resistance.

In one embodiment of the invention, a medical instrument includes a channel, a first interacting structure, a slider, and a control member. The first interacting structure can extend along at least a portion of the channel. At least a portion of the slider can also be disposed in the channel, so as to move in the channel. The slider can additionally have a first attraction portion. The control member can be rotatably coupled to the slider, and have a second interacting structure and a second attraction portion. The second interacting structure can engage the first interacting structure so as to move the slider in the channel when the control member is rotated. The second attraction portion can be disposed such that a distance between the second attraction portion and the first attraction portion changes as the control member is rotated relative to the slider.

In another embodiment of the invention, a medical instrument for receiving an attachment having a fixed portion and a moveable portion is provided. The medical instrument can include a handle, a slider, and a controller. The handle can be configured to receive the fixed portion of the attachment and have a channel. At least a portion can be disposed so as to move within the channel. Further, the slider can be configured to receive the movable portion of the attachment and have at least one magnetic portion. The controller can have at least one magnetic portion disposed so as to create a magnetic force with the at least one magnetic portion of the slider. Further, the controller can be configured such that movement of the controller causes movement of the slider, up to a threshold force at which the magnetic force is overwhelmed and the slider and the controller separate.

In a further embodiment of the invention, a medical instrument for operating a tool can include a track and a slider. The track can have a plurality of magnetic portions disposed thereto at a plurality of different locations along the track. The slider can be disposed on the track and have at least one magnetic portion disposed generally adjacent the track. The slider can move along the track such that when the slider slides through a full range of motion along the track the magnetic portions of the track and the magnetic portion of the slider are brought into a plurality of generally adjacent positions. This can affect a magnetic force between the magnetic portions of the track and the magnetic portion of the slider.

In an additional embodiment a kit can include a medical instrument and a plurality of magnetic portions. The medical instrument can have a moveable portion operatively connected to an operating portion of the medical instrument to control the operating portion. The medical instrument can also have a plurality of magnetic portions that provide at least one of an audible or tactile feedback to a user. Further, at least one of the magnetic portions can be removable and replaceable by one of the plurality of magnetic portions.

In yet another embodiment, a medical instrument can include an instrument and a first member. The instrument can include an operating portion and a non-operating portion, the non-operating portion comprising at least one first magnetic portion. The first member can include a moveable rod, a slot, and a release member. The moveable rod can include a second magnetic portion in magnetic connection with the first magnetic portion. The moveable rod can be housed with in the slot, allowing movement through the slot away from the first magnetic portion. The release member can be in operative communication with the moveable rod, such that actuation of the release member causes movement of the rod away from the first magnetic portion. This movement away from the first magnetic portion can release the magnetic connection with the first magnetic portion.

In an additional embodiment, a medical instrument can include an operating portion, a handle, and a magnet. The handle can be operatively connected to the operating portion and include at least one control piece that is moveable relative to the handle. The magnet can be disposed on at least one of the handle and the control piece so as to provide audible and/or tactile feedback to an operator.

### BRIEF DESCRIPTION OF THE DRAWINGS

These and other features, aspects, and advantages of the medical devices disclosed herein are described below with reference to the drawings of preferred embodiments, which are intended to illustrate and not to limit the invention. Additionally, from figure to figure, the same reference numerals have been used to designate the same components of an illustrated embodiment. Like components between the illustrated embodiments are similarly noted as the same reference numbers with a letter suffix to indicate another embodiment. The following is a brief description of each of the drawings.

FIG. 1A is a perspective view of an embodiment of a medical instrument configured in accordance with the present invention and includes a handle and rotary dial or control portion.

FIG. 1B is a partially exploded perspective view of a distal portion of the medical instrument of FIG. 1A with the control portion removed from the handle to show complementary magnets.

FIG. 1C is a partially exploded perspective view like that of FIG. 1B except that the control portion includes a plurality of magnets.

FIG. 1D is a partially exploded perspective view like that of FIG. 1B except that the complementary magnets are disposed on components of the control portion.

FIG. 1E is a portion of a cross-sectional view through the control portion and handle from FIG. 1A showing the complementary magnets illustrated in FIG. 1B in an aligned state.

FIG. 2A is a perspective view of another embodiment of a medical instrument configured in accordance with the present invention.

FIG. 2B is a partially exploded perspective view of a portion of the medical instrument of FIG. 2A with a control portion removed from a handle.

FIG. 2C is a perspective view of a handle of another embodiment of a medical instrument configured in accordance with the present invention that can be used with the control portion depicted in FIGS. 2A and 2B.

FIG. 2D is a side cross-sectional view of the medical instrument of FIG. 2C, taken at 2D-2D.

FIG. 3A is a partial perspective view of a portion of another embodiment of a medical instrument configured in accordance with the present invention.

FIG. 3B is a perspective view of the medical instrument of FIG. 3A with a control portion moved towards a proximal end of the handle.

FIG. 3C is a perspective view of the medical instrument of FIG. 3A with the control portion removed from the handle.

FIG. 4A is a side cross-sectional view of another embodiment of a medical instrument that includes a push drive apparatus configured in accordance with the present invention.

FIG. 4B is a side view of the medical instrument of FIG. 4A with a sliding block and fixed block of the push drive apparatus in a separated state.

FIG. 5 is a perspective view of an exemplary embodiment of a stone basket that can be attached to the medical instruments disclosed herein.

### DETAILED DESCRIPTION OF THE PREFERRED EMBODIMENTS

The present disclosure provides magnetic detent mechanisms for medical instruments. The figures depict the magnetic detent mechanisms in the context of a handle for a stone basket. More specific descriptions of various stone baskets and basket handles can be found in, e.g., U.S. Pat. Nos. 6,676,668 and 6,652,537, which are both incorporated by reference herein in their entirety. However, it will be clear from the disclosure that the magnetic detent mechanisms can include other medical and non-medical instruments. For example, the magnetic detent mechanisms can be employed with steering catheters, stents, and a variety of other medical articles. In a more general set of embodiments, the magnetic detent mechanisms are employed with any instrument for which it is desirable to provide tactile or audible feedback in addition to or in place of visible feedback, such as where an operator may be looking elsewhere or require feedback more precise than available by sight alone. In another general set of embodiments, the magnetic detent mechanisms are employed with instruments used for delicate procedures, where excessive force may cause complications, potentially causing damage to the instrument or something the instrument is acting on (such as a medical patient). In another general set of embodiments, the magnetic detent mechanisms are employed with instruments which are held in a prescribed position until released. It will be clear from the disclosure herein that the magnetic detent mechanisms can include a much greater variety of instruments than those explicitly described, medical or otherwise.

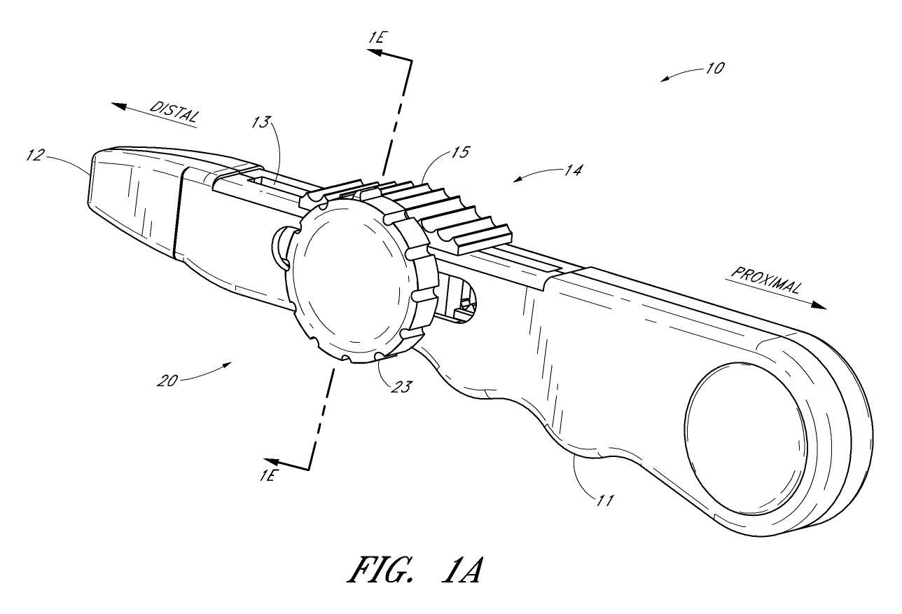

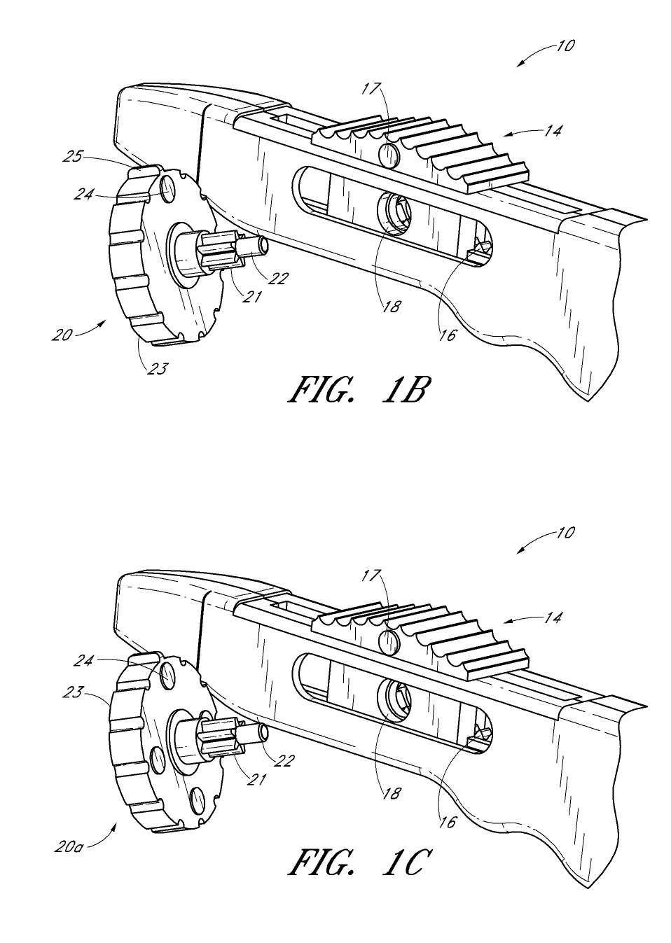

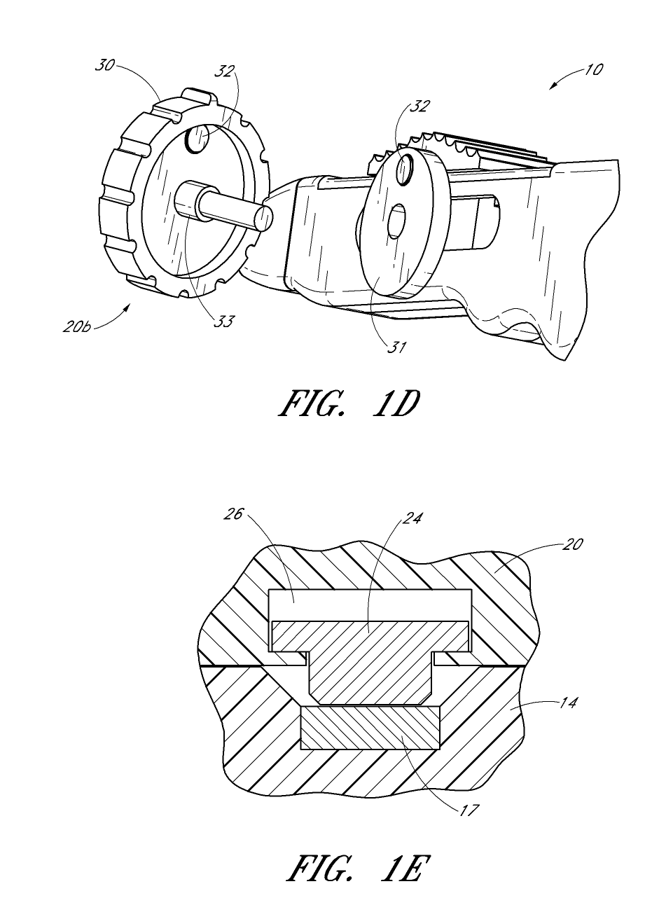

For example, an embodiment of a stone basket 1, which can be attached to a basket handle 10 that includes a magnetic detent mechanism as is illustrated in FIG. 1A, is depicted in FIG. 5. As shown, the basket 1 comprises basket wires 2, a control wire 3, and a sheath 4. The basket and control wires 2, 3 can connect and pass through the sheath 4. When the control wire 3 is pushed into the sheath 4 the basket 1 can expand as the basket wires 2 flex outward. When the control wire 3 is withdrawn, the basket 1 can close as the basket wires 2 fold. Depending for example on what is carried by the basket 1, it may be desirable to reliably control the force with which the basket opens or closes, how far the basket opens or closes, how fast the basket opens or closes, etc. Although the magnetic detent mechanism is primarily described herein in the context of using the basket handle 10 and stone basket 1, other embodiments are possible. It should generally be understood that the control wire 3 can act as a non-operating portion of the basket 1, while the basket wires 2 act as an operating portion. In further embodiments, operating and non-operating portions can take different forms.

FIGS. 1A and 1B depict one embodiment of a basket handle 10. The basket handle 10 can include a hand grip 11 and a wire entry portion 12. A slider 14 residing in a slot or channel 13 of the handle 10, depicted as a track, can hold a control wire 3 of the basket 1. As the slider 14 moves back in the slot 13 in a proximal direction toward the hand grip 11 (as indicated in FIG. 1A), the slider pulls the control wire 3 causing the basket wires 2 to close at a point distal from the basket handle 10. Similarly, movement of the slider 14 in the other or distal direction causes the basket 1 to open. In non-basket embodiments, movement of the slider 14 may cause a stent to open, a sheath to pass over a needle, or many other desired actions.

As depicted, in this embodiment movement of the slider 14 can be caused in two distinct ways. First, the slider 14 can be moved directly by a finger of an operator. The slider 14 may further include one or more ridges 15 on its upper surface to increase traction with the finger. However, the slider 14 can also move via the control portion 20. As most clearly shown in FIG. 1B, the handle 10 also include a gear track 16. The control portion 20 can include a gear 21 annularly disposed on an axle 22. The axle 22 passes through a slider hole 18 into the slot 13, allowing the gear 21 to engage with the gear track 16. As the control portion 20 rotates, it can move forward or backward at a controlled rate via the gear 21 and gear track 16. As shown, the axle 22 and hole 18 can have varying widths configured to ensure both a secure fit and adequate clearance for the gear 21. While it is preferable that the slider 14 be movable in more than one way, in some embodiments the slider 14 may only move in a single way such as with the control portion 20.

As depicted, at least one of the control portion 20 and the slider 14 comprises a magnet 17, 24. A magnet is defined as a material that produces a magnetic field. The other one of the control portion 20 and the slider 14 can comprises either a magnet 17, 24, a non-magnetized ferromagnetic material, or a paramagnetic material. For ease of explanation, the term magnet is used throughout the disclosure to include materials that produce a magnetic field, non-magnetized ferromagnetic materials, and paramagnetic materials.

The magnet 24 on the slider 14 can be positioned directly above the center of the slider hole 18 at a distance approximately the same as the distance the magnet 17 on the control portion 20 is from the center of the axle 22. However, it will be clear that the magnets 17, 24 need not be at these exact locations such as being directly above the slider hole 18. For example, the magnets 17, 24 can be an equal radial distance from the hole 18 or axle 22. As the control portion 20 rotates, so does the magnet 24, bringing it eventually into alignment with the magnet 17. Similarly, the magnets 17, 24 can be at different radially distances, since they need not be in perfect alignment to have a mutual magnetic force.

When the magnets 17, 24 approach each other, their attractive or repulsive force can increase, providing a tactile response to an operator. This force can further cause the slider 14 to naturally tend to stay (or stay away from) a given position. In some embodiments this position can be configured to coincide with, a preferred state, such as a preferred size opening of the stone basket 1. Further, the magnets 17, 24 can be chosen to have an attractive force strong enough to hold the slider 14 at the chosen position without application of some dislodging force greater than a force expected in normal operation that preferably would not cause the slider 14 to move.

To indicate this position to the operator, the control portion 20 can further include a tactile indicator 25 (depicted as a larger ridge in FIG. 1B) among ridges 23 indicating when the control portion 20 is the desired position. The magnets 17, 24 can also align at multiple positions along the slot 13, potentially defining multiple intervals that can correspond to a given distance of significance to the operator. For example, in some embodiments the control portion 20 can be configured to provide a tactile feedback at every inch of travel of the slider 14.

FIG. 1C depicts another embodiment of a control portion 20a. In this embodiment, the control portion 20a comprises multiple magnets (or magnetic materials) 24. Accordingly, the control portion 20a provides multiple tactile feedback forces in a single rotation, allowing potentially higher fidelity feedback. Further, in some embodiments the magnetic force can vary with each magnet 24, indicating an angular position of the control portion 20a.

FIG. 1D depicts another embodiment of a control portion 20b. In this embodiment, the control portion 20b includes a handle 30 and a clutch 31, each including attractive clutch magnets 32. When combined, the handle 30 and the clutch 31 can be identical to the previous control portions 20, 20a (although the outward facing magnet 24 may or may not be distinct from the clutch magnets 32).

As depicted the handle 30 and clutch 31, when combined are still free to spin independently via the smooth axle 33 of the handle 30. However, the clutch magnets 32, when aligned, can provide an attractive force inhibiting the relative rotation of the handle 30 and the clutch 31. When the handle 30 is rotated by an operator, the entire control portion 20b can rotate, causing the slider 14 to move. However, if this rotation requires too much force, the force resisting rotation of the clutch 31 can overwhelm the attractive force between the clutch magnets 32 and cause the handle 30 and the clutch 31 to rotate independently. The attractive force between the clutch magnets 32 can thus be chosen to prevent an excessive force in opening and closing the basket 1. Further, an operator can feel the sudden change in resistance as the clutch magnets 32 come out of alignment, indicating that the magnets 32 have released and that a threshold force has been reached. As discussed further below, in some embodiments the strength of the magnetic force can be adjusted to fit individual needs.

If the outer control portion magnet 24 is also present on the control portion 20b, e.g. on the clutch 31, the operator can feel the additional tactile feedback previously discussed. In some embodiments the outer magnet 24 can be the same magnet as the clutch magnet 32 on the clutch 31. In such embodiments, the tactile feedback magnetic force can be less than the clutch magnetic force.

FIG. 1E depicts an enlarged cross-sectional view of an embodiment of a control portion 20 like that in FIG. 1A, taken at 1E-1E. As depicted, the control portion 20 forms a recess 26 holding the magnet 24. Further, the magnet 24 and the recess 26 can optionally interengage to prevent the magnet from exiting the recess. The recess 26 can also have a depth greater than at least a portion of the magnet 24, allowing the magnet 24 to move inward or outward within the recess. A corresponding magnet 17 on the slider 14 can be fixed within the slider at a recessed, beveled position.

As depicted in FIG. 1E, when the magnets 17, 24 align, the magnet 24 on the control portion 20 can be attracted in an outward direction toward the magnet 17. Their contact can create a clicking sound, providing audible feedback to the operator. The beveled edges on the slider 14 and magnet 24 can then allow the magnets 17, 24 to slide out of alignment after clicking. Other embodiments providing similar functionality are possible. For example, the recess 26 can be open such that the magnet 24 can escape, but also include a weaker magnet in its interior pulling the magnet back into the recess when not overpowered by the slider magnet 17. As another example, a similar opposing force can be provided with springs.

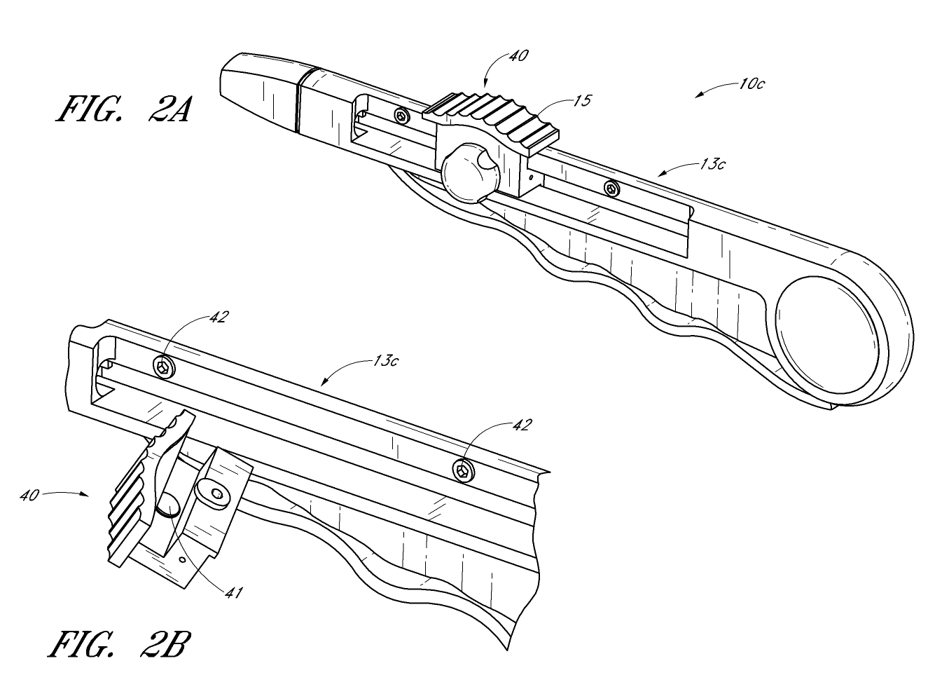

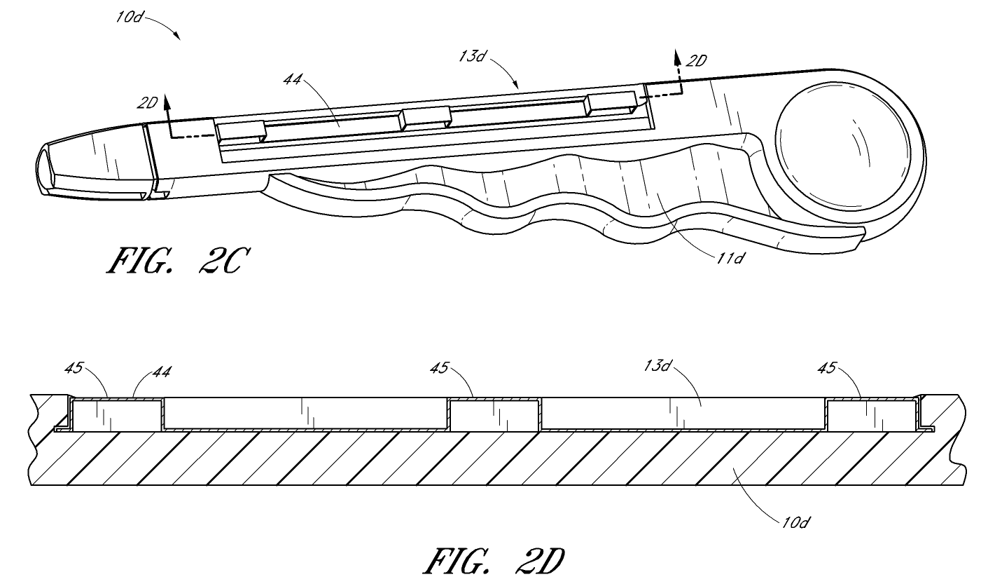

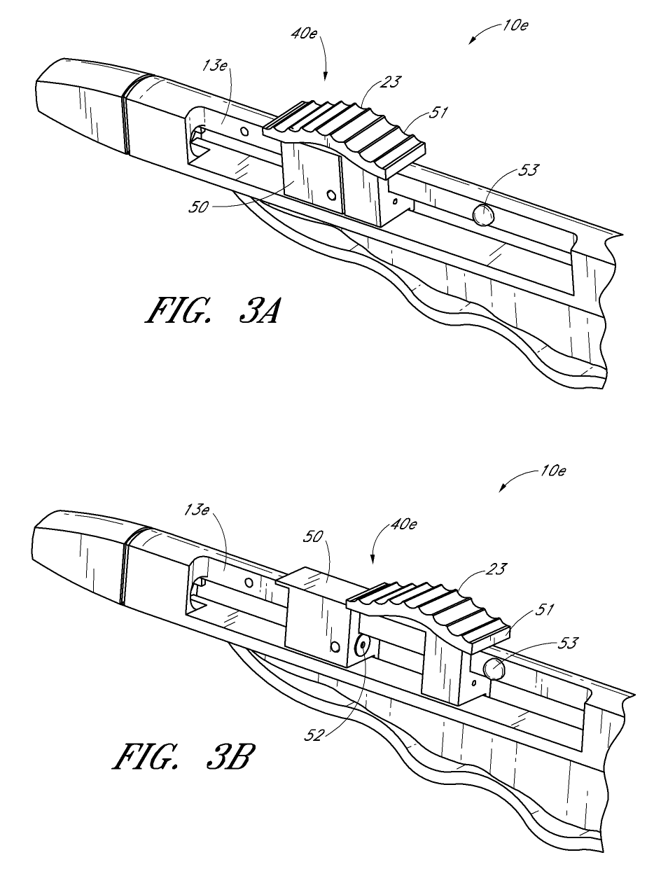

FIGS. 2A, 2B depict another embodiment of a basket handle 10c. In this embodiment, a slider 40 rides in a slot 13c and holds a control wire 3. The slider 40 can include ridges 15 and a magnet 41. The slot 13c can include one or more magnets 42. Thus, as the slider 40 moves along the slot 13c an operator can receive tactile feedback as the slider 40 moves past the slot magnets 42. As depicted here, these slot magnets 42 can indicate a starting and stopping position for the control wire 3. As discussed previously, one of either the slider magnet 41 and the slot magnets 42 may comprise a non-permanent magnet.

Further, as depicted, the slot magnets 42 can be in the form of pins or screws. An operator can easily adjust the depth of the pins or screws to modify the attractive force between the magnets 41, 42. Similarly, the slider magnet 41 can have a threaded portion with a screw head allowing its depth to be varied, or for it to be removed and replaced with a magnet of a different strength. These variations can affect both the tactile feedback and the tendency for the slider 40 to stay near or away from the slot magnets 42, as discussed above.

FIG. 2C depicts another embodiment of a basket handle 10d. The handle 10d can be used with the slider 40 depicted in FIGS. 2A, 2B. In this embodiment, the slider 40 interacts with a metal sheet 44 along the slot 13d. The metal sheet 44 can include one or more raised portions 45, at which the force from the magnet 41 on the slider 40 will be strongest due to the decreased distance between the magnet 41 and the metal sheet 44. Accordingly, the metal sheet 44 can provide similar functionality as the magnets 17, 24, 42 discussed above.

Advantageously, the metal sheet 44 can be easily replaced and reconfigured. For example, the slot 13d can have small slits at its ends, into which ends of the metal sheet 44 can be inserted, thus holding the sheet in place and allowing easy removal, as depicted in FIG. 2D. Thus a variety of magnetic strength profiles can be applied by using different metal sheets 44.

As depicted in the embodiment in FIG. 2C, the handle 10d can have a wavy hand grip 11d, with horizontal waves. The wavy hand grip 11d can provide additional tactile indications of position for the operator. As depicted, the wavy hand grip 11d includes three maxima and three minima, but other combinations or patterns are possible. In some embodiments the profile can include a short wavelength (high frequency) wave superimposed on a long wavelength (low frequency) wave, providing varying precisions on a unitary surface. In additional exemplary embodiments, the profile can be different on each side of the handle 10d.

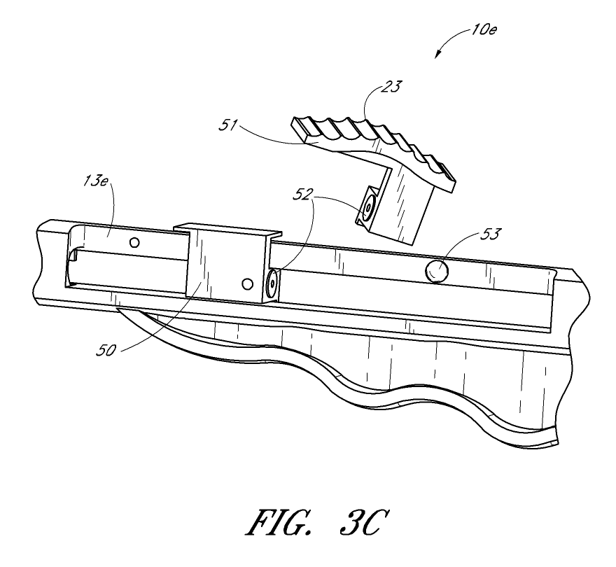

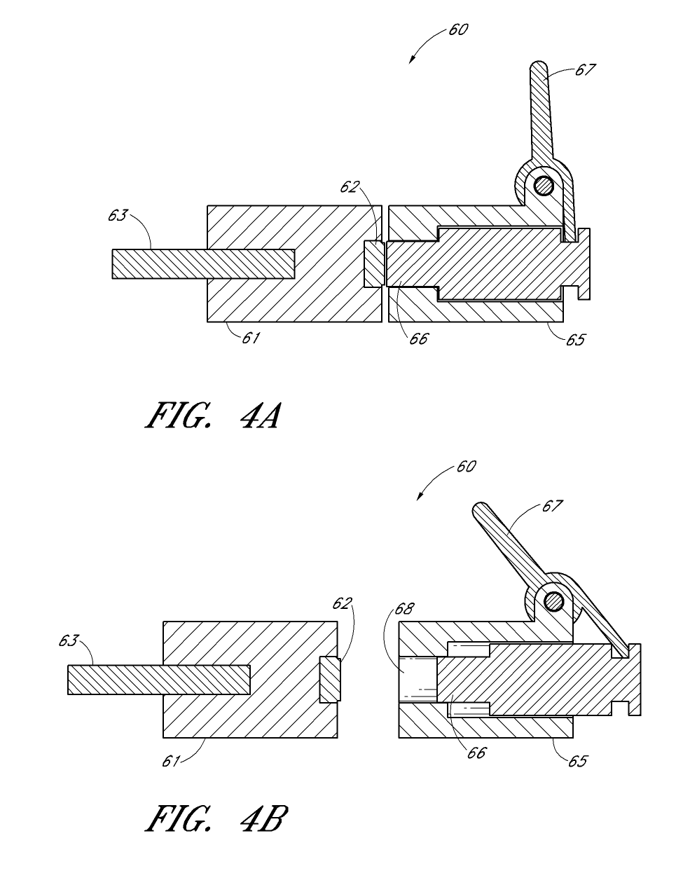

FIGS. 3A, 3B, 3C depict another embodiment of a basket handle 10e. Although slot magnets 42 and a metal sheet 44 are not depicted, it will be clear that these elements could be included. It will also be clear that the wavy features on the hand grip 10e can be similar to those on handle 10d. As depicted, the basket handle 10e includes a multi-piece slider 40e. For example, the slider 40e can include a wire holder 50 and a control piece 51. The wire holder 50 can attach to the control wire 3 and the control piece 51 can magnetically attach to the wire holder 50. The control piece 51 may further include finger ridges 23. Both the wire holder 50 and the control piece 51 can ride in the slot 13e. The slot 13e can further include a removable pin stop 53, adjustably limiting the travel of the slider 40e. As shown, each piece of the slider 40e can include a magnet 52, but as discussed above both need not be magnets.

Via the magnetic attachment, a force moving the control piece 51 in a rearward or proximal direction can similarly move the wire holder 50 in the same direction, moving the control wire 3 as well. If the force required is too great, the wire holder 50 and the control piece 51 can separate, as the magnetic attractive force is overcome. Further as discussed above, the magnets 52 can be chosen to require a specific desired force of separation, such as a force that would cause a controlled medical article to break or that would potentially cause damage to an object on which the medical article is acting.

It should also be noted that in the embodiment of FIGS. 3A, 3B, 3C, the magnetic separation occurs when moving in the proximal or rearward direction. It will be clear that in those embodiments where the control piece 51 is placed on the other side of the wire holder 50 that the separation will occur when moving in the opposite direction. Further, in some embodiments control pieces 51 can be placed on both sides, allowing the operator to have force-limited control in both directions. Similarly, if the magnets 52 are located on a non-axial side of the wire holder 50 (i.e. a side non-perpendicular to the slot 13e) and the control piece 51 does not abut a perpendicular side, then the control piece 51 can release in both directions.

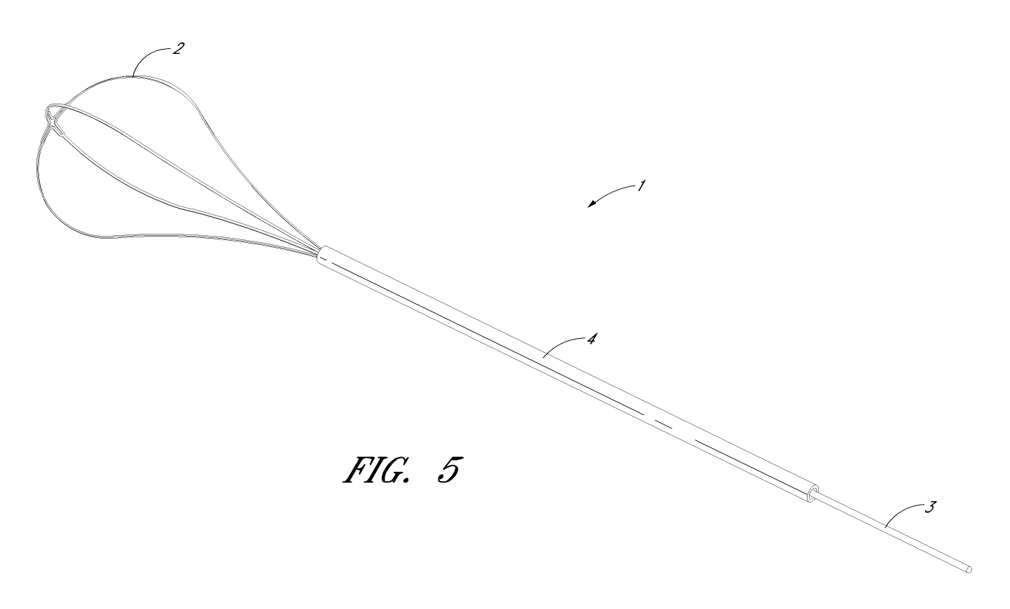

FIGS. 4A, 4B depict another embodiment of a medical instrument. The medical instrument in this embodiment is depicted as a push drive apparatus 60. As depicted, the push drive apparatus includes two blocks: a sliding block 61 and a fixed block 65. The sliding block 61 and the fixed block 65 can be held together by the interaction of a magnet 62 on the sliding block and a magnetic yet unmagnetized attachment portion 66 (depicted as a pin) residing in a hole 68 on the fixed block 65. As discussed above in relation to other embodiments, the pin 66 can also be permanently-magnetized in other embodiments. The attractive force between the magnet 62 and the pin 66 can be sufficient to resiliently hold the blocks 60, 65 together in normal operation absent actuation of a release member 67.

The fixed block 65 can release the sliding block 61 by actuation of a release member 67, depicted as a lever. It will be clear from the disclosure herein that other release members 67 can be used, including cross pins, cams, and the like. The lever 67 can attach to or abut the pin 66, and upon actuation can move the pin away from the magnet 62 through the hole 68. As the remaining material of the blocks 61, 65 prevent the magnet 62 from coming any closer to the withdrawn pin 66, the magnetic attraction can weaken sufficiently to allow separation of the blocks. The sliding block 61 can thus move away from the fixed block 65, allowing an extension 63 to perform a desired function.

Referring back to the embodiments previously discussed, the embodiment in FIGS. 4A, 4B can be integrated with a variety of medical instruments. Using the basket handles 10 as an example, the push drive apparatus 60 can prevent the movement of a slider 14 prior to actuation of a release member 67. Of course, the push drive apparatus 60 and similar embodiments can be used with other medical instruments, as discussed above.

In conjunction with the invention and the embodiments described above, a variety of materials can be used. For permanent magnets, materials such as iron, neodymium boron iron, samarium cobalt, magnetite, and cobalt can be used. For unmagnetized, magnetic materials, the invention can use steel, iron, other paramagnetic and ferromagnetic materials, by way of example. The magnetic properties can further be modified by adjusting alloys, sizes, distances between magnets, and other properties. In some embodiments, the materials can be chrome or nickel plated to improve resistance to corrosion.

In further embodiments, a medical instrument can come in the form of a kit. The magnets can reversibly insert into the medical instrument, for example by a screw-thread mechanism. Accordingly, a variety of magnets can be interchanged to modify the attractive forces. Further, via the screw-thread arrangement the magnetic force can be more finely calibrated by rotating through the thread, modifying the precise position of the magnets.

In some embodiments, a practitioner can test a medical instrument, directly or indirectly observing the magnetic force. If the magnetic force is too strong or weak by at least a given amount, the practitioner can interchange the magnets. If the magnetic force is not too strong or weak by at least the given amount, the practitioner can adjust the position of any of the magnets to more finely tune the magnetic force.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. Thus, it is intended that the scope of the present invention herein disclosed should not be limited by the particular disclosed embodiments described above, but should be determined only by a fair reading of the claims that follow.

What is claimed is:

1. A medical instrument comprising:
a channel;
a first interacting structure extending along at least a portion of the channel;
a slider having a first attraction portion, at least a portion of the slider being disposed so as to move in the channel; and
a control member being rotatably coupled to the slider and having a second interacting structure, the second interacting structure engaging with the first interacting structure so as to move the slider in the channel when the control member is rotated, and a second attraction portion disposed such that a distance between the second attraction portion and the first attraction portion changes as the control member is rotated relative to the slider.

2. A medical instrument according to claim 1, wherein the first interacting structure is a track.

3. A medical instrument according to claim 1, wherein the second interacting structure is a gear.

4. A medical instrument according to claim 1, wherein there is an attractive force between the attraction portions that varies with their distance.

5. A medical instrument according to claim 4, wherein the first attraction portion is a permanent magnet.

6. A medical instrument according to claim 4, wherein the second attraction portion is a permanent magnet.

7. A medical instrument according to claim 4, wherein at least one of the attraction portions are free to move in at least one direction within a cavity, such that its movement can create an audible feedback when generally adjacent the other attraction portion.

8. A medical instrument for receiving an attachment having a fixed portion and a movable portion, the instrument comprising:
a handle configured to receive the fixed portion and having a channel;
a slider configured to receive the movable portion and having at least one magnetic portion, at least a portion of the slider being disposed so as to move within the channel; and
a controller having at least one magnetic portion disposed so as to create a magnetic force with the at least one magnetic portion of the slider, the controller being configured such that movement of the controller causes movement of the slider up to a threshold force at which the magnetic force is overwhelmed and the slider and the controller separate.

9. A medical instrument according to claim 8, wherein the controller is rotatable relative to the slider.

10. A medical instrument according to claim 8, wherein the controller is disposed to move within the channel.

11. A medical instrument according to claim 8, wherein movement of the movable portion causes the attachment to perform an action on an object, the action having a predetermined maximum attachment force such that applying a force greater than the maximum attachment force to the attachment can damage the attachment or the object, and the threshold force is approximately equal to the maximum attachment force.

12. A medical instrument according to claim 8, wherein the channel is oriented in a proximal/distal direction relative to a grasping portion of the handle, and the slider separates from the controller when moving in a proximal direction.

13. A medical instrument according to claim 8, wherein the track is oriented in a proximal/distal direction relative to a grasping portion of the handle, and the slider separates from the controller when moving in a distal direction.

14. A medical instrument according to claim 8, wherein the threshold force is a force at which either the medical instrument or an object the instrument is acting on would be damaged if further force were applied.

15. A medical instrument for operating a tool comprising:
a track comprising a plurality of magnetic portions disposed on the track at a plurality of different locations along the track; and
a slider disposed along the track and comprising at least one magnetic portion disposed generally adjacent the track, at least a portion of the slider being disposed so as to move along the track such that when the slider slides through a full range of motion along the track the magnetic portions of the track and the magnetic portion of the slider are brought into a plurality of generally adjacent positions to affect a magnetic force between the magnetic portions of the track and the magnetic portion of the slider.

16. A medical instrument according to claim 15, wherein the magnetic portions allow a selectable magnitude of the magnetic force between the magnetic portions of the track and the magnetic portion of the slider.

17. A medical instrument according to claim 16, wherein the magnetic portions on the track comprise threaded portions that allow for the adjustment of the positions of the magnetic portions within the track.

18. A medical instrument according to claim 17, wherein the threaded portions are removably and replaceably mounted to the track.

19. A medical instrument according to claim 16, wherein the magnetic portions comprise raised portions on at least one magnetic strip removably and replaceably mounted to the track.

20. A kit comprising:
a medical instrument comprising a movable portion operatively connected to an operating portion of the medical instrument to control the operating portion, the medical instrument further comprising a plurality of magnetic portions providing at least one of an audible or tactile feedback to a user, wherein at least one of the magnetic portions is removable; and
a plurality of magnetic portions that can attach to the medical instrument to replace the removable magnetic portion.

21. A medical instrument comprising:
an instrument comprising an operating portion and a non-operating portion, the non-operating portion comprising at least one first magnetic portion;
a handle member comprising:
a moveable rod comprising a second magnetic portion in magnetic connection with the first magnetic portion;
a slot housing the moveable rod and allowing movement therethrough away from the first magnetic portion; and
a release member in operative communication with the moveable rod such that actuation of the release member causes movement of the rod away from the first magnetic portion, releasing the magnetic connection with the first magnetic portion.

22. A medical instrument handle comprising:
an operating portion;
a handle operatively connected to the operating portion and including at least one control piece that is moveable relative to the handle; and
a magnet disposed on at least one of the handle and the control piece so as to provide audible and/or tactile feedback to an operator.
